(19) United States
(12) Patent Application Publication
(10) Pub. No.: US 2024/0148977 A1
(43) Pub. Date: May 9, 2024

(54) Drug Administration Management System and Operation Method Thereof

(71) Applicant: LG CHEM, LTD., Yeongdeungpo-gu (KR)

(72) Inventors: Jong Won Lim, Yuseong-gu Daejeon (KR); Hye Won LEE, Yuseong-gu Daejeon (KR); Hyoung Seok KIM, Yuseong-gu Daejeon (KR); Soo Bum PARK, Yuseong-gu Daejeon (KR); Sang Hoon OH, Yuseong-gu Daejeon (KR)

(21) Appl. No.: 18/280,870

(22) PCT Filed: Mar. 8, 2022
(86) PCT No.: PCT/KR2022/003303

§ 371 Date: Sep. 7, 2023

(30) Foreign Application Priority Data
Mar. 9, 2021 (KR) 10-2021-0031022

(51) Int. Cl. A61M 5/315; G16H 20/17; A61M 5/31

(52) U.S. Cl. CPC A61M 5/31546; A61M 5/31568; G16H 20/17; A61M 2005/3126

(57) ABSTRACT

A drug administration management system according to an embodiment of the present disclosure may include a smart case configured to accommodate a drug administration device, display drug administration information including whether or not a drug is administered and a drug administration dose for each drug administration site input button based on an input of a user, and transmit the drug administration information externally; and a terminal configured to receive the externally transmitted drug administration information from the smart case, and monitor the drug administration information based on a drug administration plan input from a user.

### CROSS-REFERENCE TO RELATED APPLICATIONS

This application is a national phase entry under 35 U.S.C. § 371 of International Application No. PCT/KR2022/003303 filed Mar. 8, 2022, which claims priority to and the benefit of Korean Patent Application No. 10-2021-0031022 filed in the Korean Intellectual Property Office on Mar. 9, 2021, the entire contents of which are incorporated herein by reference.

### TECHNICAL FIELD

Embodiments of the present disclosure relate to a drug administration management system and an operation method thereof.

### BACKGROUND ART

For various purposes, such as treatment of various diseases or administration of weight loss or growth hormone, the number of cases of periodic self-administration of liquid drugs is increasing. In order to show the effect of self-administration of these drugs, it is important to administer the correct dose of the drug at a set time. However, these self-administration injections merely perform the function of setting and injecting only the administration dose, and it is difficult for non-expert users to manage the administration dose and time.

In addition, if the user does not record the administration dose and time individually while administering the drug, the user may forget whether the drug is administered or not. As such, if the user does not accurately identify whether the user had administered the drug or not, the drug administration dose and time, and the like, it is difficult to fully realize the effect of the drug, and in some cases, side effects may occur.

### DISCLOSURE

### Technical Problem

Embodiments disclosed herein aim to provide a drug administration management system that can support a user's efficient management of drug administration information, such as an administration site and dose of a drug, and an operation method thereof.

The technical problems of the embodiments of the present disclosure are not limited to the technical problems mentioned above, and other technical problems not mentioned will be clearly understood by those skilled in the art from the following description.

### Technical Solution

A drug administration management system according to an embodiment of the present disclosure may include a smart case configured to accommodate a drug administration device, display drug administration information including whether or not a drug is administered and a drug administration dose for each drug administration site input button based on an input of a user, and transmit the drug administration information externally; and a terminal configured to receive the externally transmitted drug administration information from the smart case, and monitor the drug administration information based on a drug administration plan input from a user.

In one embodiment, the terminal may be configured to set the drug administration plan including a drug administration date and a drug administration time of a user based on a user input, set the drug administration dose and the drug administration site of a user, and transmit a set value related to the drug administration dose and the drug administration site of a user to the smart case.

In one embodiment, the terminal may be configured to store at least one of: (i) the drug administration information received from the smart case, and (ii) a drug administration information obtained based on the user input.

In one embodiment, the terminal may be configured to compare a preset drug administration plan with the drug administration information received from the smart case or the drug administration information obtained based on the user input, and monitor at least one of: (i) whether or not a drug administration of a user is complete and (ii) a drug administration rate.

In one embodiment, the terminal may be configured to classify and monitor at least one of (i) whether or not the drug administration of a user is complete and (ii) the drug administration rate for each drug administration site of a user.

In one embodiment, the drug administration device may be configured to administer growth hormone, and the terminal may be configured to monitor a change in growth record data including at least one of a height and weight of a user according to the administration of the growth hormone.

In one embodiment, the smart case may be configured to automatically transmit the drug administration information stored in the smart case to the terminal by performing synchronization with the terminal when the smart case is turned on, and the terminal may be configured to transmit a transmission confirmation code including date data to the smart case.

In one embodiment, the smart case may be configured to receive and store the transmission confirmation code including the date data from the terminal, and delete the drug administration information previously transmitted to the terminal.

In one embodiment, the smart case may include an input unit configured to receive the input from a user, a display configured to display the drug administration information based on the input from the user, and a transceiver configured to transmit the drug administration information to the terminal.

A method for operating a drug administration management system according to an embodiment disclosed in this document may include the steps of receiving, from a user, an input of drug administration information including (i) whether or not a drug is administered and (ii) a drug administration dose for each drug administration site, displaying the drug administration information, and transmitting the drug administration information externally, receiving the externally transmitted drug administration information, and monitoring the drug administration information based on a drug administration plan input from a user.

In one embodiment, the method may further include setting a drug administration plan including a drug administration date and a drug administration time of a user based on a user input, and transmitting a set value related to the drug administration dose and the drug administration site of a user.

In one embodiment, displaying the drug administration information includes displaying (i) whether or not the drug is administered and (ii) the drug administration dose for each drug administration site.

In one embodiment, the method may further include comparing the drug administration plan to the drug administration information to monitor at least one of: (i) whether or not a drug administration of a user is complete and (ii) a drug administration rate.

In one embodiment, the method may further include classifying and monitoring at least one of (i) whether or not the drug administration of a user is complete and (ii) the drug administration rate for each drug administration site of a user.

### Advantageous Effects

According to a drug administration management system and its operation method according to an embodiment of the present disclosure, the user can efficiently manage drug administration information such as the administration site and dose of the drug through a smart case that can be linked with a drug administration management application.

### DESCRIPTION OF DRAWINGS

FIG. 1 is a block diagram illustrating the configuration of a drug administration management system according to an embodiment of the present disclosure.

FIG. 2 is a block diagram illustrating the configuration of a smart case according to an embodiment of the present disclosure.

FIG. 3 is a diagram exemplarily illustrating a smart case according to an embodiment of the present disclosure.

FIG. 4a is a diagram illustrating an operation of setting a drug administration plan at a terminal according to an embodiment of the present disclosure.

FIG. 4b is a diagram illustrating an input operation of drug administration information at a terminal according to an embodiment of the present disclosure.

FIG. 4c is a diagram illustrating the recording of drug administration information at a terminal according to an embodiment of the present disclosure.

FIG. 4d is a diagram illustrating a monitoring operation of drug administration information at a terminal according to an embodiment of the present disclosure.

FIG. 5 is a diagram illustrating the operation of a smart case and a user terminal according to an embodiment of the present disclosure.

FIG. 6 is a flowchart illustrating a method for operating a drug administration management system according to an embodiment of the present disclosure.

FIG. 7 is a block diagram illustrating a hardware configuration of a computing system configured to drive a drug administration management system according to an embodiment of the present disclosure.

### MODE FOR INVENTION

Hereinafter, some embodiments of the present disclosure will be described in detail with reference to exemplary drawings. In adding reference numerals to the components of each drawing, it should be noted that the same components are given the same reference numerals as much as possible even though they are indicated on different drawings. In addition, in describing the embodiments of the present disclosure, if it is determined that a detailed description of a related known configuration or function interferes with the understanding of the embodiment of the present disclosure, the detailed description thereof will be omitted.

In describing the components of the embodiments of the present disclosure, terms such as first, second, A, B, (a), (b), etc. may be used. These terms are only for distinguishing the component from other components, and the essence, order, or sequence of the component is not limited by the term. In addition, unless otherwise defined, all terms used herein, including technical or scientific terms, have the same meaning as commonly understood by one of ordinary skill in the art to which the embodiments of the present disclosure belong. Terms such as those defined in a commonly used dictionary should be interpreted as having a meaning consistent with the meaning in the context of the related art, and should not be interpreted in an ideal or excessively formal meaning unless explicitly defined in this disclosure.

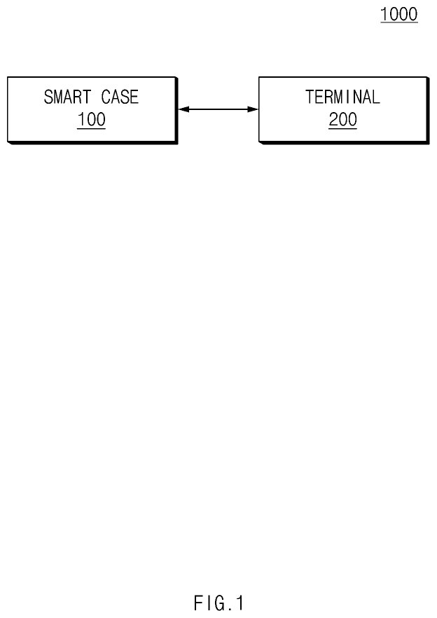

FIG. 1 is a block diagram illustrating the configuration of a drug administration management system according to an embodiment of the present disclosure.

Referring to FIG. 1, a drug administration management system 1000 according to an embodiment of the present disclosure may include a smart case 100 and a terminal 200.

The smart case 100 may be a case configured to accommodate a drug administration device (e.g., a syringe, etc.) for a user to administer a drug. The smart case 100 may include a button (input unit) that allows the user to directly input the dose of the drug administered through the drug administration device, the administration site of the drug, and the like, and an LED screen (display) configured to display the drug administration information.

The smart case 100 may store the drug administration information for the user when the user administers the drug through the drug administration device, and inputs the drug administration information such as the drug administration dose and the drug administration site. In addition, when the drug administration information is input by the user, the smart case 100 may perform synchronization by transmitting the drug administration information regarding the drug administration site and the drug administration dose of the user to the terminal 200.

The terminal 200 according to an embodiment of the present disclosure may be implemented as a portable device such as a smart phone, a tablet, or a wearable device, as well as other computing devices such as a laptop computer or a desktop computer. In addition, the terminal 200 may be implemented in the form of a mobile terminal such as a smart phone or an application installed and executed on a smart phone. For example, when the terminal 200 is implemented in the form of an application, it may be executed by a user of the mobile terminal to perform operations and functions described below.

The terminal 200 is a device operable by a user, and an application for managing the user's drug administration may be installed. That is, the user may receive drug administration information from the smart case 100 through an application installed in the terminal 200, and transmit set values related to the drug administration dose or drug administration site to the smart case 100.

The smart case 100 and the terminal 200 may communicate wirelessly. For example, the smart case 100 and the terminal 200 may be connected through Bluetooth, wifi, and the like. In addition, the smart case 100 may automatically perform synchronization with the terminal 200 when the smart case is turned on. When the synchronization is completed, the smart case 100 may automatically transmit the data that is not transmitted among the stored drug administration information of the user.

The terminal 200 may manage the user's drug administration plan, administration record, administration result (effect), and the like based on the drug administration information received from the smart case 100. In addition, the terminal 200 may compare the drug administration plan input from the user with the drug administration information received from the smart case 100 to monitor whether the user's drug administration is complete or not or a drug administration rate.

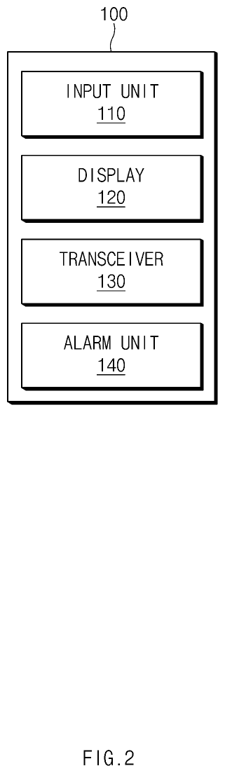

FIG. 2 is a block diagram illustrating the configuration of a smart case according to an embodiment of the present disclosure.

Hereinafter, the configuration of the smart case 100 will be described in detail with reference to FIG. 2.

The smart case 100 may accommodate the drug administration device. According to an embodiment, the smart case 100 may accommodate a drug administration device configured to administer growth hormone.

The smart case 100 may accommodate the drug administration device, store the administration information of the drug administered by the user through the drug administration device, and transmit the stored drug administration information to an external device, so that the usage history of the user's drug administration device and drug administration records can be managed.

Referring to FIG. 2, the smart case 100 may include an input unit 110, a display 120, a transceiver 130, and an alarm unit 140.

The input unit 110 may receive the drug administration information input from the user. Here, the drug administration information may include the user's drug administration site and drug administration dose. According to an embodiment, the input unit 110 may include a power button, a dose adjustment button, an administration site input button, a volume adjustment button, and a mute button. The input unit 110 may receive drug administration information including a drug administration dose and a drug administration site from the user through the dose adjustment button and the administration site input button.

The display 120 may display the drug administration information input by the user. According to an embodiment, the display 120 may display the user's drug administration site on the LED screen. Here, the user's drug administration site may be displayed as an arm, belly, thigh, hip, and the like, and whether it is left and right (L, R) for each site may be displayed together. The display 120 may basically light the drug administration site on the LED screen. In a case where the user administers the drug to a specific site, when the user touches the LED screen corresponding to the drug administration site, only the display for the corresponding site may be turned off.

The display 120 may display whether the drug is administered or not and the drug administration dose for each drug administration site input button based on the user's input. For example, in the case where the user administers a drug to the left arm, when the user touches the area indicated by the arm (L) on the LED screen of the smart case 100, only the corresponding area may be turned off. Therefore, when the user turns on the smart case 100 afterward, the user can easily see which site the drug was administered to on the previous day by looking at the blinking display of the drug administration site.

The transceiver 130 may transmit the drug administration information input from the user to the outside. For example, the external device may include a mobile phone, a tablet PC, a laptop computer, and the like.

The transceiver 130 may automatically transmit the drug administration information stored in the smart case 100 to the terminal 200 by performing automatic synchronization with the terminal 200 when the smart case is turned on.

The terminal 200 may receive and store the drug administration information from the transceiver 130, and transmit a transmission confirmation code including date data to the smart case 100.

The transceiver 130 of the smart case 100 may receive and store the transmission confirmation code including date data from the terminal 200. The smart case 100 may delete the drug administration information transmitted to the terminal 200 if the date is the same as the date of the current time in the transmission confirmation code including the date data received from the terminal.

Through this, the smart case 100 can prevent the already transmitted drug administration information from being repeatedly transmitted to the terminal 200.

The alarm unit 140 may provide the user with an alarm so that the user administers the drug at a preset time. For example, the alarm unit 140 may provide an alarm to the user through a notification sound or a guide message.

The alarm unit 140 may provide an alarm when the smart case is not turned off for a predetermined period of time. Also, the alarm unit 140 may provide an alarm to the user to administer the drug at a preset time. In this case, the alarm unit 140 may set an alarm by the user's input through the input unit 110, or may provide an alarm based on a set time received from the terminal 200. For example, the alarm unit 140 may output a guide message or a notification sound to the user. Also, the alarm unit 140 may output a warning message when synchronization with an external device is not performed for a reference time after the smart case 100 is turned on.

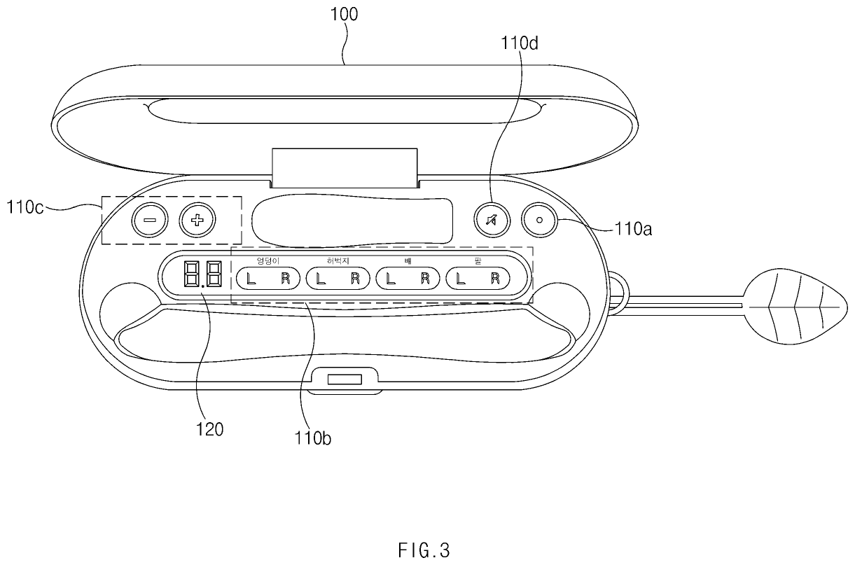

FIG. 3 is a diagram exemplarily illustrating a smart case according to an embodiment of the present disclosure.

Referring to FIG. 3, the input unit 110 of the smart case according to an embodiment of the present disclosure may include a first input unit 110a, a second input unit 110b, a third input unit 110c, and a fourth input unit 110d.

The first input unit 110a may turn on/off the smart case 100. That is, the first input unit 110a may be a power button. For example, when the user briefly presses the first input unit 110a, the smart case 100 may be turned on or off.

In addition, when the user completes the use of the smart case 100 and touches the first input unit 110a again to turn off the power, the information on the set administration dose and drug administration site is automatically synchronized with the application of the terminal 200, and the synchronized information may be transmitted to the terminal 200. In addition, the user's drug administration information may be automatically stored in a storage unit (not shown) when the smart case 100 is turned off.

As such, when the smart case 100 and the terminal 200 are synchronized by the first input unit 110a, the Bluetooth connection may be displayed on the smart case 100. For example, when synchronization is started, the smart case 100 may output a guide message saying “Synchronization (pairing) is started.”, and when synchronization is complete, the smart case may output a guide message saying “Synchronization is complete. Please set the administration dose.”, along with the Bluetooth connection sound. In addition, when the synchronization of the smart case 100 and the terminal 200 is completed, if there is data that is not transmitted to the terminal 200 among the user's drug administration information stored in the smart case 100, the corresponding drug administration information data may be automatically transmitted to the terminal 200. In this case, the smart case 100 may output, for example, a guide message saying “The drug administration site has been transmitted.” In addition, the drug administration information data transmitted to the terminal 200 may be deleted after a predetermined time.

The second input unit 110b may allow the user to input a drug administration site. In this case, the second input unit 110b may be a touch screen provided in the smart case 100. That is, when the user touches the drug administration site through the second input unit 110b after the user administers the drug to a specific site, the display on the smart case 100 corresponding to the site may be turned off. For example, when the user touches an area corresponding to the thigh (L) of the second input unit 110b after administering a drug to the right thigh, the display of the corresponding area may be turned off.

In addition, when the drug administration site is input from the user through the second input unit 110b, information on the corresponding drug administration site and drug administration dose is automatically stored in the smart case 100, and also the drug administration information may be transmitted to the terminal 200 through the smart case 100.

In this way, the user touches the drug administration site through the second input unit 110b provided in the smart case 100 to flicker the LED display, so that the user can more easily and clearly identify the site to which he/she has already administered. For example, if the user administers a drug to an arbitrary site on a specific date, the user may administer the drug to a different site from the previous day by checking whether the drug administration site displayed on the smart case 100 flickers the next day.

The third input unit 110c may allow the user to adjust the drug administration dose. That is, the user may set the desired drug administration dose by manipulating the third input unit 110c. For example, the drug administration dose may be set as an international unit (IU) value. As such, the drug administration dose set by the third input unit 110c may be displayed as a numerical value on the smart case 100.

The fourth input unit 110d may adjust or mute the volume of a warning sound or a guide message generated by the smart case 100. For example, the volume may be adjusted in stages whenever the user presses the fourth input unit 110d, and may be muted when the user presses the fourth input unit 110d for a predetermined time or longer. In addition, the fourth input unit 110d may perform synchronization with the application of the terminal 200 when the user presses the power button for a preset period of time (e.g., 2 seconds or longer).

The smart case 100 may display the drug administration dose and the drug administration site together. Referring to the left side of FIG. 3, on the smart case 100, the drug administered dose may be displayed numerically. In this case, the drug administration dose may be a value directly set by the user through the smart case 100, or may be a value set by the user at an application of the terminal 200 synchronized with the smart case 100.

In addition, the smart case 100 may display each drug administration site of the user. In this case, each drug administration site may be turned on by default, and when the user touches the site where the drug is administrated, the LED display of the corresponding site may be turned off. For example, the drug administration site on the smart case 100 may be reset again after a certain period of time (e.g., 36 hours), and LED indications of all sites may be lit. As such, the user can easily identify the site where the drug has been previously administered through the smart case 100.

In addition, the smart case 100 according to an embodiment may be configured to be automatically turned off and to transmit the administration information to the terminal 200, when the user turns off the smart case without touching the drug administration site or continues to turn on the smart case for more than a certain time without selecting the drug administration site.

In addition, in the case where the smart case 100 and the terminal 200 are not synchronized, the user's drug administration information is stored in the smart case 100 by date when the smart case is turned off or the drug administration site is selected, and then, when the smart case 100 is synchronized with the terminal 200, untransmitted data may be transmitted in batches. In this case, if the smart case 100 is not synchronized with the terminal 200 for a certain period of time (e.g., 2 weeks) or more, the alarm unit 140 of the smart case 100 may provide a warning message by itself.

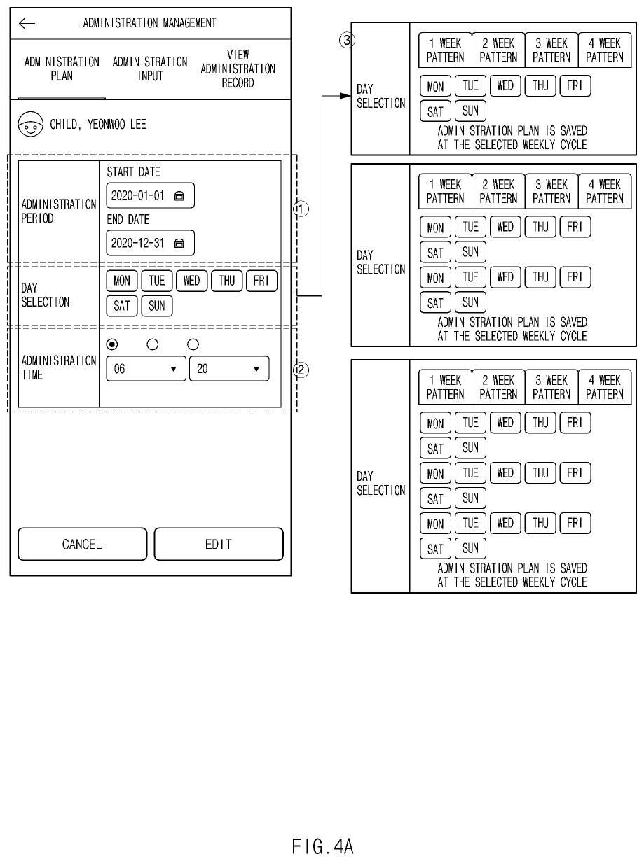

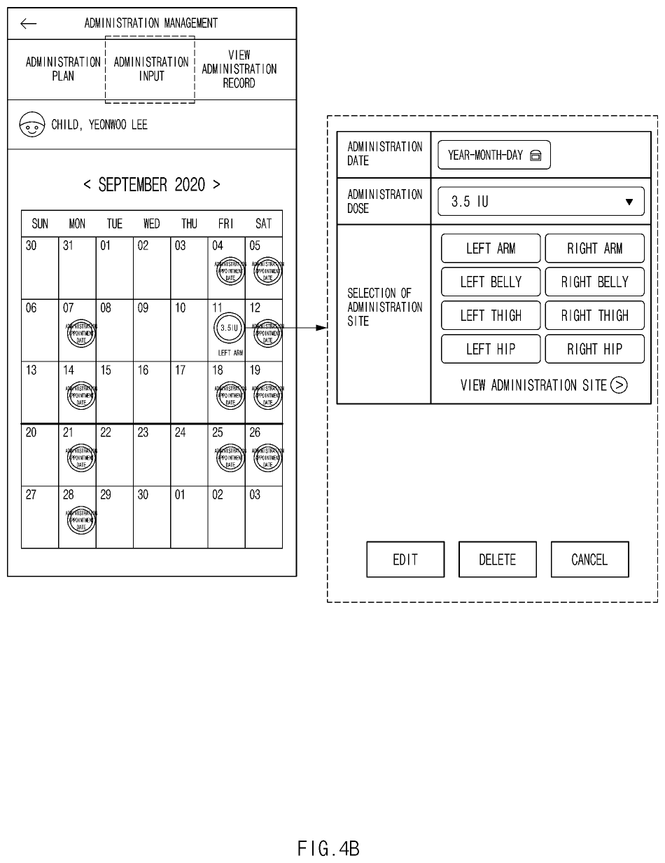

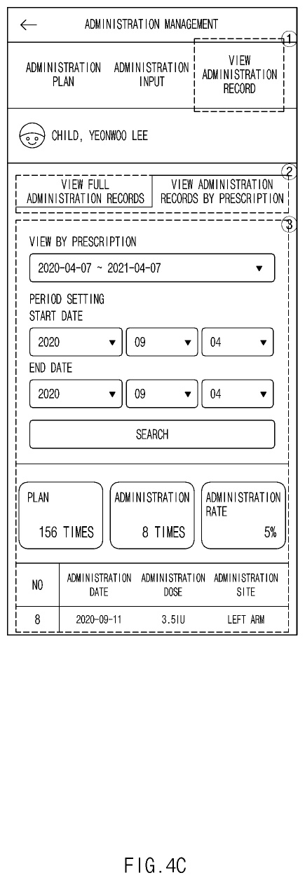

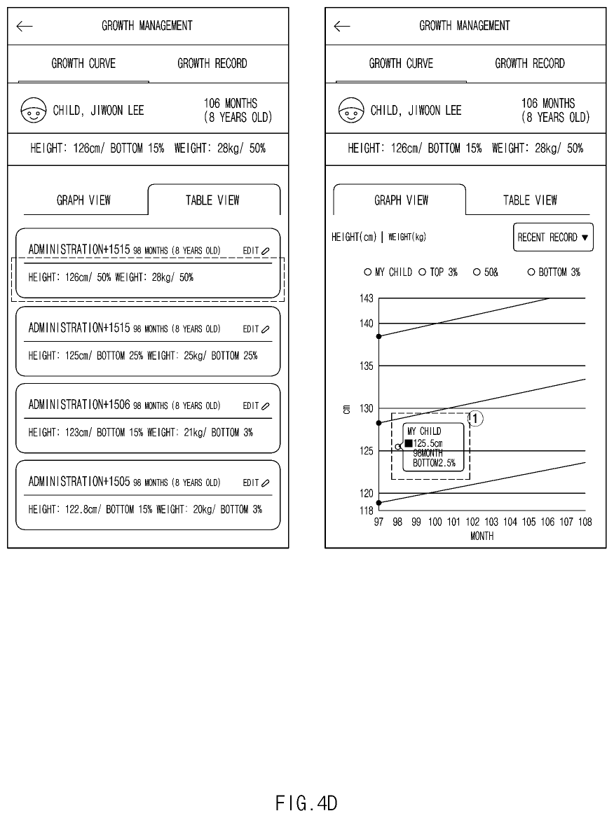

FIG. 4a is a diagram illustrating an operation of setting a drug administration plan at a terminal according to an embodiment of the present disclosure.

Referring to FIG. 4a, the terminal 200 may set a drug administration plan based on a user's input. Here, the drug administration plan may include the user's drug administration date and drug administration time. According to an embodiment, the terminal 200 may set the administration date to a period of 1 to 4 weeks based on the user's input. Also, according to an embodiment, the terminal 200 may set the drug administration plan including a drug administration dose and a drug administration site based on the user's input.

The terminal 200 may transmit the drug administration plan including the set values related to the user's drug administration dose and drug administration site to the smart case 100.

The terminal 200 may receive and store the drug administration information from the smart case 100 or may obtain and store the user's drug administration information based on the user's input.

FIG. 4b is a diagram illustrating an input operation of drug administration information at a terminal according to an embodiment of the present disclosure.

Referring to FIG. 4b, the terminal 200 may store the user's drug administration information based on the user's input. Here, the drug administration information may include the user's drug administration date, drug administration time, drug administration dose, and drug administration site. The terminal 200 may classify and store the user's drug administration dose for each drug administration site of the user.

FIG. 4c is a diagram illustrating the recording of drug administration information at a terminal according to an embodiment of the present disclosure.

Referring to FIG. 4c, the terminal 200 may output a record of the drug administration information received from the smart case 100 or a record of the user's drug administration information obtained based on the user's input.

For example, the terminal 200 may output the drug administration dose of ‘3.5 IU’ and the drug administration site of ‘left arm’ on the user's drug administration date of Sep. 11, 2020.

In addition, the terminal 200 may compare a preset drug administration plan with the drug administration information received from the smart case 100 or the drug administration information obtained based on the user's input, monitor whether the user's drug administration is complete or not and the drug administration rate, and output the monitored result.

FIG. 4d is a diagram illustrating a monitoring operation of drug administration information at a terminal according to an embodiment of the present disclosure.

The terminal 200 may compare the preset drug administration plan with the drug administration information received from the smart case 100 or the drug administration information obtained based on the user's input, and monitor whether the user's drug administration is complete or nor or a drug administration rate.

According to an embodiment, the terminal 200 may display the growth management (e.g., growth curve) and growth record (record date, height and weight records, etc.) of a child patient according to the user's growth hormone injection administration. For example, the terminal 200 may monitor a change in the growth record data including at least one of a user's height and weight according to the administration of growth hormone.

According to an embodiment, the terminal 200 may provide a guide video on a method of administering a drug, or may provide a request or consultation for items necessary for drug administration.

In addition, the terminal 200 may manage the drug administration plan, the drug administration information, the drug administration result (effect), and the like. Specifically, the terminal 200 may set the user's drug administration plan (e.g., drug administration date, drug administration time), and provide a push notification for drug administration at the set date and time.

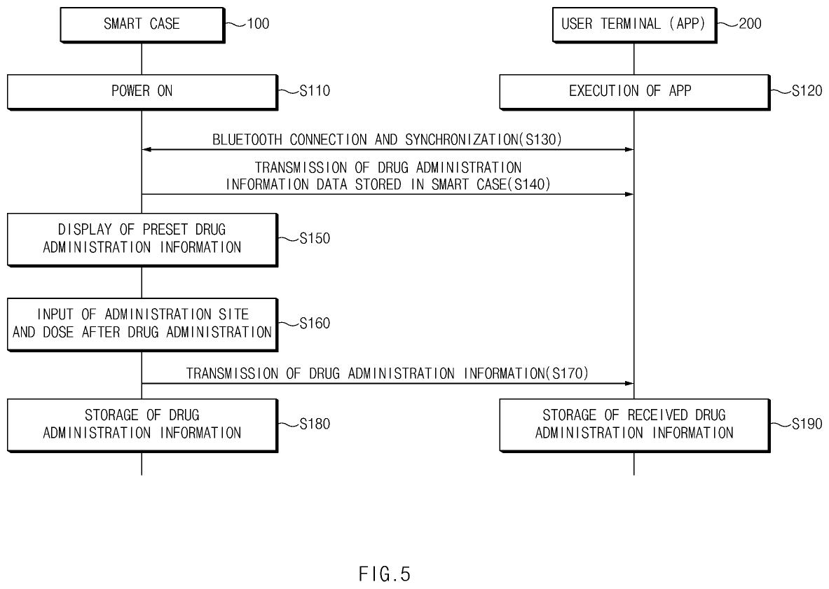

FIG. 5 is a diagram illustrating the operation of a smart case and a user terminal according to an embodiment of the present disclosure.

Referring to FIG. 5, in the step of S110, the power of the smart case 100 may be turned on by the user.

In the step of S120, the terminal 200 may execute an application.

In the step of S130, the smart case 100 and the terminal 200 may be connected through Bluetooth to perform synchronization.

In the step of S140, when the synchronization of the smart case 100 and the terminal 200 is completed, the user's drug administration information data stored in the smart case 100 may be automatically transmitted to the terminal 200.

In the step of S140, the terminal 200 may receive and store the drug administration information, and transmit the transmission confirmation code including date data to the smart case 100.

In the step of S140, the smart case 100 may receive and store the transmission confirmation code including the date data from the terminal 200. The smart case 100 may delete the drug administration information transmitted to the terminal 200 if the date is the same as the date of the current time in the transmission confirmation code including the date data received from the terminal.

In the step of S150, the smart case 100 may display the preset drug administration information, for example, the drug administration site, the drug administration dose, and the like. In the step of S150, the smart case 100 may display whether or not a drug is administered for each drug administration site, the drug administration site, the drug administration dose, and the like, based on the user's input.

In the step of S160, when the user administers the drug through the provided drug administration device, the smart case 100 may receive an input regarding the drug administration site and the drug administration dose. In the step of S160, for example, the smart case 100 may turn on or off the indication of the site where the drug is administered based on the user's input. That is, in the step of S160, the smart case 100 may turn off the display of the corresponding site when the user touches an area indicating the drug administration site after administering the drug to a specific site.

In the step of S170, the smart case 100 may automatically transmit the corresponding drug administration information to the terminal 200 when information on the administration dose and the drug administration site is input from the user.

In the step of S180, the smart case 100 may store the drug administration information.

In the step of S190, the terminal 200 may store the drug administration information received from the smart case 100. In the step of S190, the smart case 100 may also output a warning message when the synchronization with an external device is not performed for a reference time after the smart case is turned on. In the step of S190, if the smart case 100 is not turned off for more than a threshold time, a warning sound may be output.

As described above, in the smart case and its operation method according to an embodiment of the present disclosure, the user can efficiently manage the drug administration information such as the drug administration site and the drug administration dose through the smart case capable of being linked with the drug administration management application,

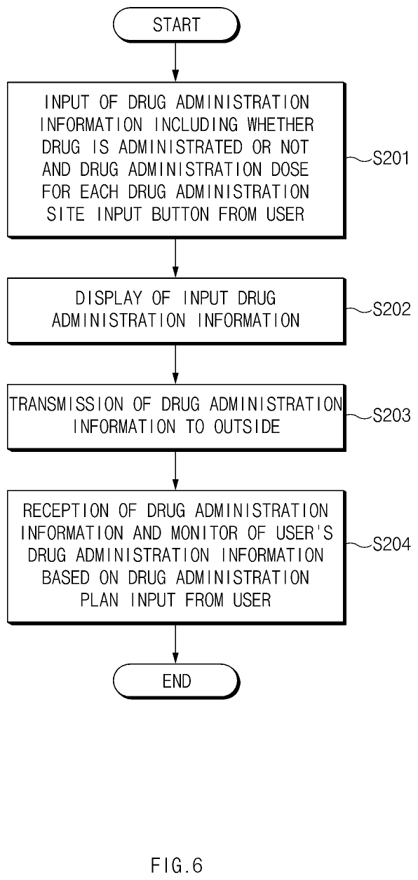

FIG. 6 is a flowchart illustrating a method for operating a drug administration management system according to an embodiment of the present disclosure.

Hereinafter, a method for operating the drug administration management system 1000 will be described with reference to FIGS. 1 to 5.

Since the smart case 100 and terminal 200 of the drug administration management system 1000 may be substantially the same as the smart case 100 and the terminal 200 described with reference to FIGS. 1 to 5, a description is briefly provided below to avoid duplication of description.

Referring to FIG. 6, the method for operating the drug administration management system according to an embodiment of the present disclosure may include the steps of receiving an input of the drug administration information including whether a drug is administrated or not and the drug administration dose for each drug administration site input button from the user (S201), displaying the input drug administration information (S202), transmitting the drug administration information to the outside (S203), and receiving the drug administration information and monitoring the user's drug administration information based on the drug administration plan input from the user (S204).

In the step of S201, the terminal 200 may set the drug administration plan including the user's drug administration date and drug administration time based on the user's input, and transmit the set values related to the user's drug administration dose and drug administration site.

In the step of S201, the terminal 200 may receive an input from the user. In the step of S201, the smart case 100 may receive and obtain the input of the drug administration information such as the drug administration dose or the drug administration site from the user. Specifically, in the step of S201, the smart case 100 may receive the drug administration information including whether or not the drug is administered and the drug administration dose for each drug administration site input button from the user.

In the step of S202, the smart case 100 may display the drug administration information based on the input from the user.

In the step of S202, the smart case 100 may display whether or not the drug is administered and the drug administration dose for each drug administration site based on the user's input.

In the step of S202, for example, the smart case 100 may turn on or off the display of the site where the drug is administrated when the drug administration site is input from the user. In particular, in the step of S201, the smart case 100 may basically display the drug administration site on the LED screen. Here, the drug administration site includes arms, stomach, thighs, hips, and the like, and the right or left side may also be displayed for each site. In this case, the drug administration site displayed on the smart case 100 may be configured to be turned on or off when the user touches it.

In the step of S203, the smart case 100 may transmit the drug administration information to an external device. In the step of S203, when the smart case 100 is turned on, the synchronization with the terminal 200 may be performed automatically. In this case, when the smart case 100 synchronizes with the terminal 200, the stored drug administration information may be automatically transmitted to the terminal 200. In the step of S203, the user's previous drug administration information (e.g., drug administration dose, drug administration site, etc.) may be updated on the application of the terminal 200.

In the step of S203, the smart case 100 may receive and store the transmission confirmation code including the date data from the terminal 200. In the step of S203, the smart case 100 may delete the drug administration information transmitted to the terminal 200 if the date is the same as the date of the current time in the transmission confirmation code including the date data received from the terminal.

In the step of S204, the terminal 200 may compare the drug administration plan with the drug administration information and monitor whether or not the user's drug administration is complete or the drug administration rate.

In the step of S204, the terminal 200 may compare the preset drug administration plan with the drug administration information received from the smart case 100 or the drug administration information obtained based on the user's input, and monitor and output whether or not the user's drug administration is complete and the drug administration rate.

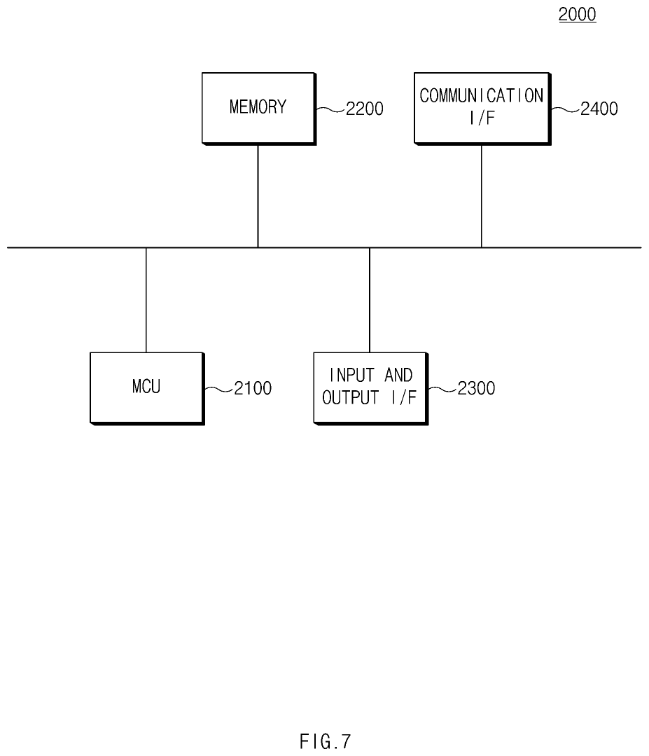

FIG. 7 is a block diagram illustrating a hardware configuration of a computing system configured to drive the drug administration management system according to an embodiment

Referring to FIG. 7, a computing system 2000 according to an embodiment of the present disclosure may include a microcontroller unit (MCU) 2100, a memory 2200, an input and output interface (I/F) 2300, and a communication I/F 2400.

The MCU 2100 executes various programs (e.g., input and transmission program of drug administration information, alarm program, etc.) stored in the memory 2200, and processes various data through these programs, and it may be a processor that performs the functions of the drug administration device shown in FIG. 2.

The memory 2200 may store various programs related to the input and transmission of the drug administration information. In addition, the memory 2200 may store various data such as the administration dose, the drug administration site, and the like.

A plurality of such memories 2200 may be provided as needed. The memory 2200 may be a volatile memory or a non-volatile memory. As the volatile memory 2200, random access memory (RAM), dynamic random access memory (DRAM), static random access memory (SRAM), and the like may be used. As the non-volatile memory 2200, read-only memory (ROM), programmable read-only memory (PROM), electronically alterable read-only memory (EAROM), erasable programmable read-only memory (EPROM), electrically erasable programmable read-only memory (EEPROM), flash memory, and the like may be used. Examples of the above-listed memories 2200 are merely examples and are not limited to these examples.

The input and output I/F 2300 may provide an interface that connects between an input device (not shown) such as a keyboard, mouse, and touch panel and an output device such as a display (not shown), and the MCU 2100 to transmit/receive data.

The communication I/F 2400 is a configuration capable of transmitting and receiving various data from an external device (e.g., a user terminal), and may be various devices capable of supporting wired or wireless communication. For example, through the communication I/F 2400, a set value or various data related to the drug administration dose or the drug administration site may be transmitted/received to/from an external device provided separately.

In this way, the computer program according to an embodiment of the present disclosure is recorded in the memory 2200 and processed by the MCU 2100, so that it may be implemented as a module that performs each function shown in FIG. 2, for example.

The above description is merely illustrative of the technical spirit of the present disclosure, and various modifications and variations will be possible without departing from the essential characteristics of the present disclosure by those of ordinary skill in the art to which the present disclosure pertains.

Accordingly, the embodiments disclosed in the present disclosure are for explanation rather than limiting the technical spirit of the present disclosure, and the scope of the technical spirit of the present disclosure is not limited by these embodiments. The protection scope of the present disclosure should be interpreted by the following claims, and all technical spirits within the scope equivalent thereto should be construed as being included in the scope of the present disclosure.

1. A drug administration management system comprising:
a smart case configured to accommodate a drug administration device, display drug administration information including whether or not a drug is administered and a drug administration dose for each drug administration site input button based on an input of a user, and transmit the drug administration information externally; and
a terminal configured to receive the externally transmitted drug administration information from the smart case, and monitor the drug administration information based on a drug administration plan input from a user.

2. The drug administration management system according to claim 1, wherein the terminal is configured to set the drug administration plan including a drug administration date and a drug administration time of a user based on a user input, set the drug administration dose and the drug administration site of a user, and transmit a set value related to the drug administration dose and the drug administration site of a user to the smart case.

3. The drug administration management system according to claim 2, wherein the terminal is configured to store at least one of: (i) the drug administration information received from the smart case, and (ii) a drug administration information obtained based on the user input.

4. The drug administration management system according to claim 3, wherein the terminal is configured to compare a preset drug administration plan with the drug administration information received from the smart case or the drug administration information obtained based on the user input, and monitor at least one of: (i) whether or not a drug administration of a user is complete and (ii) a drug administration rate.

5. The drug administration management system according to claim 4, wherein the terminal is configured to classify and monitor at least one of (i) whether or not the drug administration of a user is complete and (ii) the drug administration rate for each drug administration site of a user.

6. The drug administration management system according to claim 1, wherein the drug administration device is configured to administer growth hormone, and wherein the terminal is configured to monitor a change in growth record data including at least one of a height and weight of a user according to the administration of the growth hormone.

7. The drug administration management system according to claim 1, wherein the smart case is configured to automatically transmit the drug administration information stored in the smart case to the terminal by performing synchronization with the terminal when the smart case is turned on, and
wherein the terminal is configured to transmit a transmission confirmation code including date data to the smart case.

8. The drug administration management system according to claim 7, wherein the smart case is configured to receive and store the transmission confirmation code including the date data from the terminal, and delete the drug administration information previously transmitted to the terminal.

9. The drug administration management system according to claim 1, wherein the smart case includes an input unit configured to receive the input from a user, a display configured to display the drug administration information based on the input from a user, and a transceiver configured to transmit the drug administration information to the terminal.

10. A method for operating a drug administration management system comprising the steps of:
receiving, from a user, an input of drug administration information including (i) whether or not a drug is administered and (ii) a drug administration dose for each drug administration site input button;
displaying the drug administration information;
transmitting the drug administration information externally;
receiving the externally transmitted drug administration information;
monitoring the drug administration information based on a drug administration plan input from a user.

11. The method according to claim 10, further comprising:
setting a drug administration plan including a drug administration date and a drug administration time of a user based on a user input, and
transmitting a set value related to the drug administration dose and the drug administration site of a user.

12. The method according to claim 11, wherein displaying the drug administration information includes displaying (i) whether or not the drug is administered and (ii) the drug administration dose for each drug administration site.

13. The method according to claim 12, further comprising comparing the drug administration plan to the drug administration information to monitor at least one of: (i) whether or not a drug administration of a user is complete and (ii) a drug administration rate.

14. The method according to claim 13, further comprising classifying and monitoring at least one of (i) whether or not the drug administration of a user is complete and (ii) the drug administration rate for each drug administration site of a user.

15. The method according to claim 10, further comprising monitoring a change in growth record data including at least one of a height and a weight of a user according to an administration of a growth hormone.

16. The drug administration management system according to claim 2, wherein the drug administration device is configured to administer growth hormone, and wherein the terminal is configured to monitor a change in growth record data including at least one of a height and weight of a user according to the administration of the growth hormone.

17. The drug administration management system according to claim 3, wherein the drug administration device is configured to administer growth hormone, and wherein the terminal is configured to monitor a change in growth record data including at least one of a height and weight of a user according to the administration of the growth hormone.

18. The drug administration management system according to claim 4, wherein the drug administration device is configured to administer growth hormone, and wherein the terminal is configured to monitor a change in growth record data including at least one of a height and weight of a user according to the administration of the growth hormone.

19. The drug administration management system according to claim 5, wherein the drug administration device is configured to administer growth hormone, and wherein the terminal is configured to monitor a change in growth record data including at least one of a height and weight of a user according to the administration of the growth hormone.
